(19) United States
(12) Patent Application Publication
(10) Pub. No.: US 2023/0233782 A1
(43) Pub. Date: Jul. 27, 2023

(54) SYSTEM FOR CONTROL AND RESPIRATORY FUNCTION MAINTENANCE

(71) Applicants: Evgeniy Aleksandrovich EvGOREMYKIN, Krasnogorsk (RU); Igor Aleksandrovich LAVROV, Saint Petersburg (RU)

(72) Inventors: Evgeniy Valerievich Grudovich, Minsk (BY); Erdzhanik Grachiyaevna Abramyan, Cheboksary (RU)

(21) Appl. No.: 18/008,875

(22) PCT Filed: Jun. 21, 2021
(86) PCT No.: PCT/RU2021/050173

§ 371 Date: Dec. 7, 2022

(30) Foreign Application Priority Data
Jun. 25, 2020 (RU) 2020121136

(51) Int. Cl. A61M 16/00; A61N 1/36

(52) U.S. Cl. CPC A61M 16/024; A61N 1/36031; A61M 2205/505; A61M 2205/3569; A61M 2230/43; A61M 2230/46; A61M 2230/42; A61M 2230/08; A61M 2205/054

(57) ABSTRACT

The invention relates to medicine, The technical result of the present invention is to increase the efficiency of treatment of the respiratory system pathologies. The system includes a communication unit connected to a ventilator, a human-machine interface unit, a data processing, storage and management unit, an algorithmic unit, a pulse generation unit, a unit of electromyography electrodes and a stimulation.

### FIELD OF THE INVENTION

The invention relates to the field of medicine, namely to devices that are part of the respiratory control complex for patients in need of mechanical ventilation. The control complex is a system consisting of a pulses generation device, integrated with the systems of mechanical ventilation (ventilator or BiPAP).

### BACKGROUND OF THE RELATED ART

The following solutions are known in the conventional art.

A device for mechanical ventilation of the lungs comprising stimulation of the intercostal muscles, a diaphragm stimulator, responding to periodic signals to stimulate the work of the diaphragm, and a control unit connected to the stimulators to generate a first periodic signal and a second periodic signal, so as to establish the simultaneous movement of the intercostal muscles and a diaphragm is known (U.S. Pat. No. 5,678,535).

A device for manipulating exhalation is also known. It includes an implantable electrode capable of implantation, to stimulate the muscle connected to the respiration and to cause the reaction of the diaphragm; and electrical stimulator connected to implantable electrode and programmed by the protocol of electrical stimulation directed to increase the functional residual capacity of the patient's lungs; the stimulator has an electrical communication with the implantable electrode to thereby send stimulating electrical signal according to the electrical stimulation protocol (U.S. Pat. No. 9,259,573).

The disadvantage of the solutions mentioned above is that they do not provide the possibility of data exchange with systems for lungs artificial ventilation applied in severe respiratory diseases and that they do not consider initiation and support of the rhythmic respiratory movement with activation of relevant critics in the spinal cord.

The closest analogue of the proposed solution is a device that includes an electric signal generator, electrodes implanted in the patient's diaphragm for applying electrical stimulation to the diaphragm. The generator is a four-channel device that allows to independently control the amplitude, frequency, time and pulse widths (U.S. Pat. No. 8,406,885). It is proposed to use this device for generating pulses simultaneously with mechanical artificial ventilation of the lungs, synchronizing the stimulating pulses with the phases of breathing using a ventilator, for this the pulse generator is connected to the sensors of respiration, pressure, etc.

The disadvantage of the known technical solution is the focus on peripheral stimulation and the lack of feedback between the pulse generator and the ventilator, as well as the lack of control of the functional state of the muscular apparatus, which entails a low efficiency in solving the problem of negative side effects manifested in patients who have undergone or are undergoing mechanical ventilation.

### SUMMARY OF THE INVENTION

The technical problem solving by the present invention is the elimination of the adverse side effects caused by invasive mechanical ventilation, such as:
development of infectious complications,
because the patients with chronic and acute respiratory disorders have to remain connected to the ventilator much longer, pressure and an increase in the strength, quantity and rate of oxygen supply leads to the lung damage and limits blood oxygen saturation,
the alveoli damage in some cases differs from the acute respiratory disorders (ARDS) (70-80% in Italy), also, in spite of the ventilator, the oxygen concentration in the blood remains low,
development of weakness and atrophy of the respiratory muscles after several days of being on mechanical ventilation,
atrophy and reduced contractibility of the diaphragm,
barotrauma of the lungs.

The main object of the invention is to develop both for the patient and medical personnel a safe technology of breathing control in patients with acute and chronic breathing disorders, and particularly in cases acute viral or bacterial origin or following chronic CNS injury or dysfunction. For example, COVID-19, bilateral pneumonia, tuberculosis, and also in cases of chronic respiratory disorders during trauma of the spinal cord and, chest trauma, hypoventilation syndrome, craniocerebral trauma with signs of respiratory insufficiency of varying degrees of severity, critically lowering of the backup respiration, respiratory inefficiency (pathological condition when the respiratory minute volume is higher than 151/min., and at normal or a slightly elevated CO2 does not achieve adequate saturation of arterial blood with oxygen), etc. due to non-invasive or minimally invasive neuro stimulation (hereinafter referred to as: system or system “Neurolungs” or complex or “ Neurolungs” complex).

The technical result of the present invention is to increase the efficiency of the respiratory system pathologies treatment, achieved by the restoration and maintenance of respiratory function in patients undergoing artificial ventilation procedures. It is reached by evaluation of the optimal mode of stimulating effects on the patient's neuromuscular apparatus until the specified (normative) parameters of respiratory events for each patient are achieved by exchanging data between the stimulating pulse generation device with the ventilator to develop required movements of the part of muscle groups responsible for breathing,

The combination of stimulation of the respiratory muscles and stimulation of the spinal segments that regulate breathing will be covered by stimulation of the following areas:
Internal and external intercostal muscles:
Scalenus muscles;
Serratus dorsalis inspirator and expiratory muscles;
Abdominal muscles:
Iliocostalis muscle;
Musculus trans versus thoracis:
Diaphragm;
Cervical segments of the spinal cord;
Thoracic segments of the spinal cord.

The technical result is achieved by the system design to maintain respiratory function by neuro stimulation of the patients on mechanical lungs ventilation. The system includes a communication unit connected to a ventilator, a human-machine interface unit, data processing, storage and management unit, an algorithmic unit, a pulse generation unit, a unit of electromyography electrodes and stimulation (EMG unit). A communication unit is connected to the ventilator includes a communication interface capable to receive the data on the parameters of the ventilator and sensors that determine the amount of carbon dioxide, the amount of oxygen, volume of the air entering the lungs, the frequency of inspiration and expiration, and to transmit data to the control unit of the ventilator on pulse generating device parameters. A human-machine interface unit contains data input means for a pulse generation unit control and information output means. A data processing, storage and management unit is associated with ensuring the receipt and transmission of data with a communication unit connected to a ventilator and a human-machine interface unit, as well as with an algorithmic unit, the input of which is connected to the output of the EMG unit, and output sent to a pulse generation unit connected with the unit of stimulation electrodes. The algorithmic unit is configured to synchronize the data received from sensors of EMG unit and the output data transmitted to the pulse generation unit.

In particular cases of invention implementation, data on the ventilator parameters include the amount of carbon dioxide, the amount of oxygen in the patient's body, the volume of air entering the lungs (respiratory volumes), the frequency of inhalation and exhalation.

In particular cases of invention development the data on parameters of generating device operation include parameters characterizing the operation of stimulating electrodes.

In particular cases of invention embodiment, the data generator parameters include parameters characterizing data received from EMG system.

In particular cases of invention embodiment, the data on generator operation parameters include parameters characterizing the parameters of generating device settings, statistical parameters and operating mode parameters.

The possibility of EMG registering and the data of the protocol of artificial lung ventilation allows to organize the procedure for monitoring the respiratory function by electro stimulation of the muscles involved in spontaneous breathing with maximum efficiency. Registration of EMG, control of breathing parameters and synchronization of stimulating effects in accordance with respiratory events (inhalation, exhalation, holding of breath) provides an improvement in the quality of treatment of respiratory failure and a reduction in the period of rehabilitation after the termination of procedures for mechanical ventilation of the lungs. Meanwhile, the patient's functional state muscle control (based on the recorded EMG signals), gives the possibility of choosing the optimal amplitude stimulating effects on muscles and control of ventilation mode to select both the amplitude and rate of electroneurostimulation (frequency, duration of the signal) received by the patient during the rehabilitation procedure. The data received from external sensors containing information on the modes of operation of ventilators, as well as from EMG sensors containing information about the patient's muscle contraction in target muscle groups, are transmitted to the data processing, storage and management unit, where the data obtained is compared with the specified parameters (determined for each patient individually depending on age, weight, height, etc.) and the parameters of the stimulation signal are calculated to achieve the correspondence between the real and specified parameters of respiration (volume, rhythm, gas content in the body). At the same time, the presence of feedback from the sensors and ventilator ensures the control of breathing parameters depending on the stimulation parameters, where, in the absence of improvement in respiratory characteristics after stimulation, the ventilator control unit sends a request to the generation device to change the stimulation parameters and/or changes the operating parameters of ventilator.

Additional features and advantages of the invention will be set forth in the description that follows, and in part will be apparent from the description, or may be learned by practice of the invention. The advantages of the invention will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE ATTACHED DRAWINGS

The accompanying drawings, which are included to provide a further understanding of the invention and are incorporated in and constitute a part of this specification, illustrate embodiments of the invention and, together with the description, serve to explain the principles of the invention.

In the drawings:

FIG. 1 the general diagram of the interaction between the components of the complex, which includes a pulse generation unit.

FIG. 2 is a structural diagram of a pulse generation unit for a respiratory monitoring system in patients connected to artificial lung ventilation devices.

FIG. 3 is a diagram of external devices connection to a human-machine interface unit.

FIG. 4 is a schematic of electrodes connection to impulses generating device for a respiratory monitoring system in patients connected to ventilators.

FIG. 5 is a schematic of electrodes connection to impulses generating device for a respiratory monitoring system in patients connected to ventilators.

FIG. 6 is a schematic of electrodes connection to impulses generating device for a respiratory monitoring system in patients connected to ventilators.

### DETAILED DESCRIPTION OF EMBODIMENTS OF THE INVENTION

Reference will now be made in detail to the preferred embodiments of the present invention, examples of which are illustrated in the accompanying drawings.

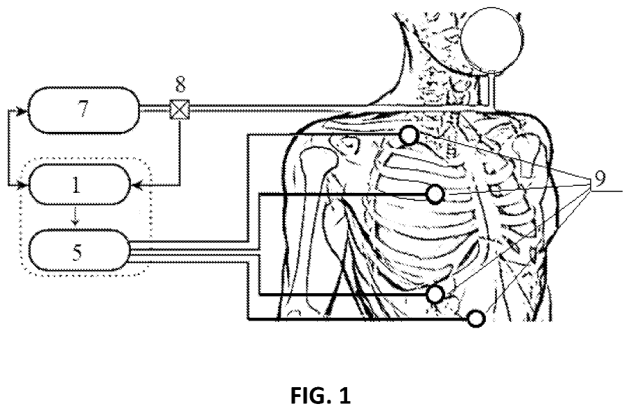

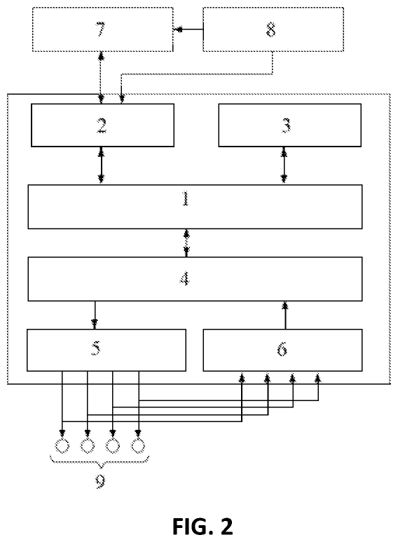

The device for generating impulses (hereinafter DGI) is a component of the “Neuro-lungs” complex. The generalized structure of interaction between the components of the “Neuro-lungs ” complex is shown on FIG. 1.

The main functional purpose of the DGI device:
Receiving information from external systems;
Transmission of information to external systems;
Formation and delivery of electrical impulses with established electrical and time characteristics to external connected electrodes;
Registration of electrical activity of muscles (8-channel EMG system);
Provision of a human-machine interface for input of tuning parameters, as well as displaying information about the operation of the complex (via the Device for Parameters Setting).

To perform these functions, the DGI was designed with the following set of units: 1—a data processing, storage and management unit; 2—a communication unit; 3—a human-machine interface unit (HMI); 4—an algorithmic unit; 5—a pulse generation unit, 6—an EMG unit. In addition, the transfer of information to external systems (the so-called “feedback”) must be ensured. The transmitted information should describe the operating modes of the DGI device and its individual components. The sets of parameters transmitted from the DGI to external systems are adjusted depending on the need and type of external device connected.

Basically, there are 3 groups of parameters:
output parameters for DGI (characterizing the operating mode of the electrodes);
input parameters for the DGI (characterizing the EMG
internal parameters of the DGI (operating mode, tuning parameters, statistical, etc.).

To ensure the synchronization function of the process of stimulation with the respiratory function, the device circuitry provides a unit for processing signals received from the ventilator. The transmission of information from external systems is implemented by using electrical interfaces (Ethernet, RS232, USB, etc.). The interface type, exchange parameters, as well as the exchange protocol are set depending on the availability of such in external systems. Communication unit 2 with external systems must contain hardware and software modules. Hardware modules are responsible for the physical implementation of the selected interface. Software modules are part of the firmware and are a set of drivers of the corresponding protocols.

Human-machine interface unit 3 (hereinafter HMI) provides an interface between a user and a “Neurolungs” complex. The control/parameters settlement/complex setting in different operation modes is accomplished using this unit. Also, this unit is used to display all the information of the “Neurolungs” complex to the user. HMI unit provides the work in two basic modes: settlement mode, monitoring mode.

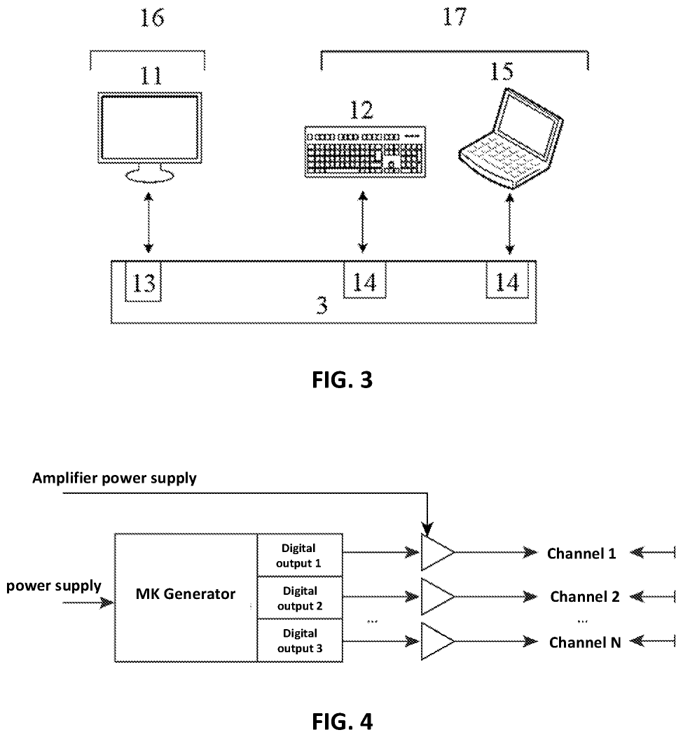

FIG. 3 schematically shows the options for using unit 3 in the described modes, where in the monitoring mode 16 unit 3 includes the information output device 11 connected via HDMI 13 interface and information input device 12 connected via USB interface 14.

The tuning/settlement mode 17 is used by the operator of the complex to enter the necessary tuning parameters, the task of operating modes for a specific selected case. This mode allows the system user (namely, the medical personnel responsible for treating the patient) to control the complex, to set the necessary parameter values. For these purposes, a personal computer PC 15 connected via the USB 14 interface under control of the OS is used (a portable/laptop PC is recommended). To perform the described actions, the Host application must be installed on the PC.

The monitoring mode is used for the process of monitoring the patient's condition, as well as the operation of the entire complex. For the settlement mode, namely for outputting information, the information output device 11 (external monitor) must be used. Connection to the DGI device is carried out through a graphical interface. Video signal is transmitted with parameters of 720p@ 50/60 Hz.

In addition, for the settlement mode, it is possible to enter (correct) the set of operating parameters in real time. To accomplish this task, it is necessary to provide for the connection of an information input device 12 via an interface (USB or other) 14.

Data processing, storage and management unit 1 provides the receiving, storing and transmitting of all data to/from the complex accomplishes the mathematical processing, computing, control of the work of all the constituent units of DGI devices. Unit 1 is a set of hardware and software tools—a computer, the main modules of which are: a microprocessor, random access memory (RAM), read-only memory (ROM), system or micro ss-board, bus controllers. Software means of unit 1: operating system; control program; built-in software (for some versions).

For the basis for the implementation of hardware can be selected baseboards with processor modules: SOM

(System On Module); ETX; microProcessor modules; PICO; SMARC or others.

Algorithmic unit 4 is responsible for implementing the algorithms of the complex, and namely for forming and issuing electrical pulses defined by electrical and timing characteristics to multiple electrode 9.

Additionally, unit 4 performs the function of receiving and processing data obtained from EMG.

This unit is a software. Separated from all other software components of the system, since designed to perform the basic algorithms of the “Neurolungs” complex.

A pulse generation unit 5 is responsible for the formation of electrical pulses with the required characteristics (amperage, voltage, frequency, flow rate, pulse shape, etc.) and is a hardware unit of the analog output stage (at least 8 channels for connecting with multiple electrode 9).

For peripheral control of respiration, impulses are used to stimulate the following muscles:

Internal and external intercostal muscles:
Scalenus muscles;
Serratus dorsalis inspirator and expiratory muscles;
Abdominal muscles;
Iliocostalis muscle;
Musculus transversus thoracis;
Diaphragm;

For facilitation and maintenance of the rhythmic respiratory activity, impulses are used to stimulate the spinal cord using non-invasive and invasive approaches:
Cervical segments of the spinal cord;
Thoracic segments of the spinal cord.

For each of these types of muscles or stimulated spinal cord segments, and the parameters of electrical impulses are different. In addition, the parameters of electrical impulses for a specific muscle group differ for each patient and are calculated each time separately depending on the age, height, weight of the patient, his general condition, the data obtained on muscle contraction during stimulation, the mode of operation of the ventilator, data from gas sensors. O₂ and CO₂ in blood (for example, optical spectrometric sensors), respiratory frequency, volume of air entering the lungs calculated using the data obtained from flow and pressure sensors.

The number of channels for connecting electrodes in the DGI device is at least 8 channels.

For each channel, it is possible to generate electrical impulses with the following characteristics:
Minimum pulse duration—1 ms;
Frequency range of filling a pulse with a modulated signal—from 100 Hz to 10 kHz (with a step of 10 Hz);
Amperage—from 20 to 150 mA.

Unit 5 is controlled by algorithmic unit 4, which sends information about the parameters of the electrical impulse. Unit 5 performs exclusively generation.

Unit 5 may contain a different composition of submodules with different functional purposes. In this case, the stimulation unit contains a unit of digital-to-analog converters, the output of each of which is connected through an amplifier with an electrode from multiple electrodes 9, through which electrostimulation is carried out and from which EMG data is taken directly during the rehabilitation procedure (FIG. 4).

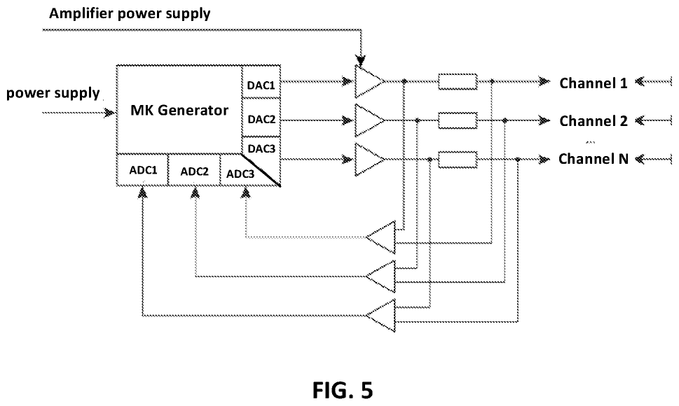

According to the circuit shown on FIG. 5, and the measuring unit includes comparators, where the connection points of each electrode are disposed on both sides of the resistor connected after the amplifier and transmits the obtained value to the algorithmic unit.

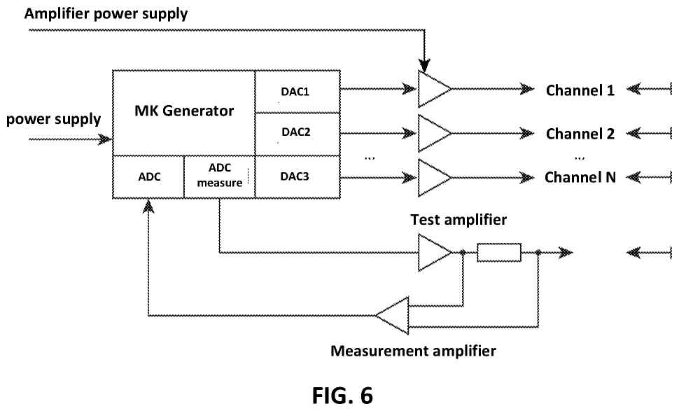

According to the scheme on FIG. 6, the measuring unit includes the unit for test signals generation, comprising data acquisition system to which the electrode is connected across the test amplifier and a measuring amplifier also connected to the electrode of the test circuit.

As sensors are used the adhesive reusable electrodes comprising a substrate of non-woven insulation material, e.g., polyethylene terephthalate, conductive element and biocompatible conductive hydrogel adhesive, and connected to the measuring and stimulating minutes chain by plugs.

Algorithmic unit 4 performs the function of EMG signal processing, digital filtering and diagnostics and as well, if necessary, and the preparation and transmission of EMG processed data, stimulation parameters for further processing and correction of stimulation parameters to the data processing, storage and management unit.

A generation device is a single housing hard- wired connected to electrodes, a control unit of the ventilator, sensors of respiratory characteristics and a human-machine interface unit. Units mentioned above are connected to a controller comprising an EMG signal processing module and a signal processing unit from sensors and a ventilator. The device operates both from a power network while the battery is being charged, and from a portable battery.

The device works as follows.

All information on the state of the device, the quality of electrode placement, data received from the ventilator, and recorded EMG signals is transmitted to the data processing, storage and management unit from the moment the device is turned on.

After placing the electrodes and turning on the device with the help of the keyboard, the device connects with EMG unit and the interelectrode impedance is estimated using an algorithmic unit. In the case of discrepancy of impedance to specified range, the error is displayed on the indicator indicating the incorrect installation of electrodes, it is recommended to reinstall the electrodes and to make the secondary evaluations of impedance.

The hardware and software unit for processing of input signals determines the connection and operability of the multiple electrode 9 for stimulation and EMG removal, indicates their operability in the information output device 14 and enables the program of the information data processing, storage and management unit to synchronize the operation of the stimulation pulse shaping unit with the EMG data using the algorithmic unit.

Next, the rhythm and volume of breathing is assessed (how optimally the lungs inhale air and exhale). In this case, the standard parameters of the rhythm and volume are taken as 100%, and the real ones are adjusted to them with the help of stimulation, by adjusting the frequency, amplitude and duration of the signal. Respiratory rhythm and volume are a more inert indicator than the data from the EMG unit, but nevertheless, the time for a sufficient collection of data is 1-2 minutes. The received data from the feedback of the ventilator and respiration sensors allow to select the optimal stimulation parameters for a given patient.

Finally, the content of gases (O₂ and CO₂) is estimated. This is the most inert parameter, the assessment of which often depends on the disease, the state of the lungs and the circulatory system. When a signal on a gas content is received that differs from the preset for a given patient, the stimulation parameters are selected to achieve 100% of the preset gas content. If, despite the stimulation, received information shows the insufficient amount of a gas content in the blood, then it is concluded that, in the current state, only the selection of stimulation parameters is not enough, and it is also necessary to additionally regulate the oxygen supply to the body.

The further mode of operation of the device is determined by the program of the rehabilitation procedure. Each program has its own set of stimulation parameter values and is designed for a specific problem. With the help of the information input device 12, it is possible to promptly control the stimulating effect (stimulation amplitude). The possibility to connect an information output device allows the doctor to observe all the information received from EMG sensors and from external systems (the ventilator and the O₂, CO₂ sensors used in it, the volume of air entering the lungs, the frequency of entry/exhalation), form complex synchronizing connections and stimulation parameters depending on the signals of the respiration sensors, the ventilator and EMG, other patient data and the results of stimulation, and, in fact, form a program for the therapeutic and rehabilitative use of the device.

Having thus described a preferred embodiment, it should be apparent to those skilled in the art that certain advantages of the described method and apparatus have been achieved.

It should also be appreciated that various modifications, adaptations and alternative embodiments thereof may be made within the scope and spirit of the present invention. The invention is further defined by the following claims.

What is claimed is:

1. A system for control and respiratory function maintenance by neurostimulation of patients on mechanical ventilation comprising:
a communication unit connected to a ventilator,
a human-machine interface unit,
a data processing, storage and management unit,
an algorithmic unit.
a pulse generation. unit,
a unit of electromyography electrodes, and
a stimulation. (EMG unit),
wherein the communication unit is connected to a ventilator through a communication interface capable of receiving data on parameters of the ventilator and sensors that determine an amount of carbon dioxide, an amount of oxygen, a volume of the air entering the lungs, and inspiration and expiration frequency, and of transmitting data to a control unit of the ventilator for parameters of the pulse generating device, and
a human-machine interface unit including a data input means for a pulse generation unit control and an information output means, and
a data processing, storage and management unit is associated with ensuring a receipt and transmission of data, with the communication unit connected to the ventilator, to the human-machine interface unit and to the algorithmic unit, and
wherein an input of algorithmic unit is connected to an output of EMG unit, and an output of algorithmic unit is connected to the pulse generation unit to control stimulation electrodes, and
wherein the algorithmic unit is configured to synchronize data received from the sensors of EMG unit and to output data transmitted to a pulse generation unit, and
wherein the pulse generation unit is configured to generate pulses to stimulate the intercostal muscles, diaphragm, abdominal muscles, cervical and thoracic spinal cord with different pulse parameters for each muscle type.

2. The system of claim 1, wherein the data processing, storage and management unit receives parameters characterizing a mode of operation of stimulating electrodes.

3. The system of claim 1, wherein parameters of the pulse generation. unit include parameters characterizing data received from a unit of electromyography electrodes and stimulation.

4. The system of claim 1, wherein data on operation of the pulse generation unit includes device settings, statistical parameters and operating mode parameters.
